(19) United States
(12) Patent Application Publication
(10) Pub. No.: US 2011/0172144 A1
(43) Pub. Date: Jul. 14, 2011

(54) METHODS FOR INCREASING EXPRESSION OF SERCA2A IN CARDIAC MUSCLE

(75) Inventor: Krisztina M. Zsebo, Del Mar, CA (US)

(73) Assignee: CELLADON CORPORATION, La Jolla, CA (US)

(21) Appl. No.: 12/984,552

(22) Filed: Jan. 4, 2011

Related U.S. Application Data
(60) Provisional application No. 61/292,437, filed on Jan. 5, 2010.

(51) Int. Cl. A61K 38/17; A61K 31/7088; C07K 14/47; C07H 21/00; C12N 15/63; A61P 9/00; A61P 9/12; A61P 9/04; A61P 9/10

(52) U.S. Cl. 514/1.2; 514/44 R; 530/400; 536/23.5; 435/320.1

(57) ABSTRACT

The present invention relates to methods for augmenting SERCA2 mediated calcium ion transport into the sarcoplasmic reticulum of cardiac myocytes of a host. The method includes administering to host cardiac myocytes a zinc finger protein that induces expression of SERCA2a (or administering a nucleic acid molecule encoding such a protein), wherein expression of SERCA2a produces an augmentation in SERCA2 mediated calcium ion transport in treated myocytes, as compared to untreated myocytes. Also provided are methods of treating heart failure by administering to a patient in need thereof, a zinc finger protein or zinc finger protein fused to an effector domain, that induces expression of SERCA2a.

### RELATED APPLICATIONS

This application claims priority under 35 U.S.C. §119(e) of U.S. Provisional application Ser. No. 61/292,437 filed Jan. 5, 2010 of which is hereby incorporated by reference in its entirety.

### BACKGROUND OF THE INVENTION

1. Field of the Invention

The invention relates generally to methods of treating heart failure, and more specifically to methods for improving cardiac contractility through increasing expression of SERCA2a using zinc finger proteins.

2. Background Information

Heart failure is a debilitating disease, in which the heart loses the ability to efficiently pump blood to meet the body's needs. This impairment of circulation deprives vital organs of oxygen and nutrients. Fatigue, weakness and the inability to carry out daily tasks may result, although not all heart failure patients suffer debilitating symptoms immediately. In general though, the disease is relentlessly progressive. As heart failure progresses, it tends to become increasingly difficult to manage. Even the compensatory responses it triggers in the body may themselves eventually complicate the clinical prognosis.

The regulation of Ca²⁺ concentration and cycling in cardiac myocytes plays an important role in many heart disorders, including heart failure. The sarcoplasmic reticulum (SR) is an internal membrane system, which plays a critical role in the regulation of cytosolic Ca²⁺ concentrations and thus, excitation-contraction coupling in muscle. Contraction is mediated through the release of Ca²⁺ from the SR, while relaxation involves the active re-uptake of Ca²⁺ into the SR lumen by a Ca²⁺-ATPase. In cardiac muscle, the SR Ca²⁺-ATPase activity (SERCA2a) is under reversible regulation by phospholamban.

Zinc finger proteins (ZFPs) are DNA-binding domains and consist of varying numbers of zinc fingers, ranging from two in the Drosophila regulator ADR1, to the more common three in mammalian Sp1 and up to nine in TFIIIA. Zinc finger proteins occur in nature as the part of transcription factors conferring DNA sequence specificity as the DNA-binding domain. They have also found use in protein engineering due to their modularity and have potential.

### SUMMARY OF THE INVENTION

In one embodiment of the present invention, there are provided methods for augmenting SERCA2 mediated calcium ion transport into the sarcoplasmic reticulum of cardiac myocytes of a host. The method includes administering to host cardiac myocytes a zinc finger protein that induces expression of SERCA2a, wherein expression of SERCA2a produces an augmentation in SERCA2 mediated calcium ion transport in treated myocytes, as compared to untreated myocytes.

In another embodiment of the invention, there are provide methods for treating or preventing a cardiovascular disorder in a patient. The methods include administering to the cardiac muscle of the patient a zinc finger protein that induces expression of SERCA2a, wherein expression of SERCA2a produces an augmentation in SERCA2 mediated calcium ion transport in treated cardiac muscle, thereby increasing cardiac muscle contractility and treating or preventing the cardiovascular disorder. In some embodiments the cardiovascular disorder is restenosis, pulmonary hypertension or a cardiac disorder, such as heart failure, ischemia, myocardial infarction, congestive heart failure, arrhythmia, transplant rejection and the like.

In certain aspects of the above embodiments of the invention, the zinc finger protein binds to a target nucleotide sequence of a nucleic acid that modulates expression of a SERCA2a gene. In one aspect, the zinc finger protein binds to a target nucleotide sequence within the promoter region of a SERCA2a gene.

In further aspects of the above embodiments of the invention, the zinc finger protein is fused to an effector domain. In some aspects, the effector domain is a transcription factor or transcription activation domain thereof. In one aspect, the transcription factor or transcription activation domain thereof is selected from the group consisting of p53, NFAT, NF-κB and VP16.

In some embodiments of the above methods of the invention, the ZFP or ZFP fused to an effector domain is co-administered with a an expression vector containing a SERCA2A gene. In one aspect, the ZFP or ZFP fused to an effector domain is administered as a nucleic acid molecule encoding the ZFP or ZFP fusion protein with the expression vector containing a SERCA2A gene. In some aspects, the nucleic acid molecule encoding the ZFP or ZFP fusion protein and the SERCA2a gene are contained within the same expression vector.

Alternatively, the nucleic acid molecule encoding the ZFP or ZFP fusion protein and the SERCA2a gene may be contained within the separate expression vectors. In one aspect, such separate expression vectors are administered simultaneously. In another aspect, the expression vectors are administered separately with the ZFP or ZFP effector domain fusion protein encoding expression vector administered first and the SERCA2a encoding expression vector administered second. In another aspect, the expression vectors are administered separately with the SERCA2a encoding expression vector administered first and the ZFP or ZFP effector domain fusion protein encoding expression vector administered second.

In another embodiment of the invention, there are provided isolated zinc finger proteins containing at least three zinc fingers that bind to a target nucleotide sequence of a SERCA2a gene promoter and induce transcription of the SERCA2a gene. In some aspects, the zinc finger protein is fused to an effector domain. In certain aspects, the effector domain is a transcription factor or transcription activation domain thereof. In one aspect, the transcription factor or transcription activation domain thereof is selected from the group consisting of p53, NFAT, NF-κB and VP16.

In still another embodiment of the invention there are provided, isolated nucleic acid molecules encoding the zinc finger proteins of the invention. In some embodiments, the nucleic acid is contained within an expression vector. In certain embodiments, the expression vector is a viral expression vector. In one aspect, the viral expression vector is an adenoviral expression vector or an AAV vector.

In a further embodiment, there are provided compositions including a ZFP that binds to a target nucleotide sequence of a SERCA2a gene promoter and induces transcription of the SERCA2a gene or such a ZFP fused to an effector domain, and an expression vector containing a SERCA2A gene. In another embodiment, there is provided a composition an expression vector containing a polynucleotide encoding a ZFP that binds to a target nucleotide sequence of a SERCA2a gene promoter and induces transcription of the SERCA2a gene or such a ZFP fused to an effector domain, and an expression vector containing a SERCA2A gene.

### DETAILED DESCRIPTION OF THE INVENTION

Before the present methods and compositions are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The present invention relates to a method for augmenting SERCA2a mediated calcium ion transport into the sarcoplasmic reticulum of cardiac myocytes of a host. The method includes administering to host cardiac myocytes a zinc finger protein that induces expression of SERCA2a, wherein expression of SERCA2a produces an augmentation in SERCA2 mediated calcium ion transport in treated myocytes, as compared to untreated myocytes.

In one embodiment, the invention features a method of treating or preventing a cardiovascular disorder in a subject. The method includes introducing into the cardiac muscle of the subject, e.g., into the heart muscle of a subject, a zinc finger protein that induces expression of SERCA2a. Expression of SERCA2a produces an augmentation in SERCA2 mediated calcium ion transport in treated cardiac muscle, thereby increasing cardiac muscle contractility and treating or preventing the cardiovascular disorder. In some embodiments, the cardiovascular disorder is restenosis, pulmonary hypertension or a cardiac disorder. In one aspect, the cardiac disorder is heart failure, ischemia, myocardial infarction, congestive heart failure, arrhythmia, or transplant rejection. In a particular aspect, the cardiac disorder is heart failure. In another aspect, the method is for the prevention of arrhythmia, restenosis, or pulmonary hypertension.

As used herein, “zinc finger protein,” “zinc finger polypeptide,” or “ZFP” refers to a polypeptide having nucleic acid, e.g., DNA, binding domains that are stabilized by zinc. The individual DNA binding domains are typically referred to as “fingers,” such that a zinc finger protein or polypeptide has at least one finger, more typically two fingers, more preferably three fingers, or even more preferably four or five fingers, to at least six or more fingers. Each finger binds from two to four base pairs of DNA, typically three or four base pairs of DNA. A ZFP binds to a nucleic acid sequence called a target nucleic acid sequence. Each finger usually comprises an approximately 30 amino acids, zinc-chelating, DNA-binding subdomain. An exemplary motif of one class, the Cys2-His2 class (C2H2 motif), is -CYS-(X)2-4-CYS-(X)12-HIS-(X)3-5-His, where X is any amino acid, and a single zinc finger of this class consists of an alpha helix containing the two invariant histidine residues and the two cysteine residues of a single beta turn that binds a zinc cation (see, e.g., Berg et al., Science, 271:1081-1085 (1996)). A zinc finger protein can have at least two DNA-binding domains, one of which is a zinc finger polypeptide, linked to the other domain via a flexible linker. The two domains can be identical or different. Both domains can be zinc finger proteins, either identical or different zinc finger proteins. Alternatively, one domain can be a non-zinc finger DNA-binding protein, such as one from a transcription factor.

As used herein, “framework (or backbone) derived from a naturally occurring zinc finger protein” means that the protein or peptide sequence within the naturally occurring zinc finger protein that is involved in non-sequence specific binding with a target nucleotide sequence is not substantially changed from its natural sequence. For example, such framework (or backbone) derived from the naturally occurring zinc finger protein maintains at least 50%, and preferably, 60%, 70%, 80%, 90%, 95%, 99% or 100% identity compared to its natural sequence in the non-sequence specific binding region. Alternatively, the nucleic acid encoding such framework (or backbone) derived from the naturally occurring zinc finger protein can be hybridizable with the nucleic acid encoding the naturally occurring zinc finger protein, either entirely or within the non-sequence specific binding region, under low, medium or high stringency condition. Preferably, the nucleic acid encoding such framework (or backbone) derived from the naturally occurring zinc finger protein is hybridizable with the nucleic acid encoding the naturally occurring zinc finger protein, either entirely or within the non-sequence specific binding region, under high stringency condition.

Zinc finger proteins designed and predicted according to the procedures in WO 98/54311 can be used in the present methods. WO 98/54311 discloses technology which allows the design of zinc finger protein domains that bind specific nucleotide sequences that are unique to a target gene. It has been calculated that a sequence comprising 18 nucleotides is sufficient to specify an unique location in the genome of higher organisms. Typically, therefore, the zinc finger protein domains are hexadactyl, i.e., contain 6 zinc fingers, each with its specifically designed alpha helix for interaction with a particular triplet. However, in some instances, a shorter or longer nucleotide target sequence may be desirable. Thus, the zinc finger domains in the proteins may contain at least 3 fingers, or from 2-12 fingers, preferably, 3-8 fingers, more preferably 5-7 fingers, and most preferably 6 fingers.

When a multi-finger protein binds to a polynucleotide duplex, e.g., DNA, RNA, PNA or any hybrids thereof, its fingers typically line up along the polynucleotide duplex with a periodicity of about one finger per 3 bases of nucleotide sequence. The binding sites of individual zinc fingers (or subsites) typically span three to four bases, and subsites of adjacent fingers usually overlap by one base. Accordingly, a three-finger zinc finger protein XYZ binds to the 10 base pair site abcdefghij (where these letters indicate one of the duplex DNA) with the subsite of finger X being ghij, finger Y being defg and finger Z being abcd. For example, as known in the art, to design a three-finger zinc finger protein to bind to the targeted 10 base site abcdefXXXX (wherein each “X” represents a base that would be specified in a particular application), zinc fingers Y and Z would have the same polypeptide sequence as found in the original zinc finger discussed above (perhaps a wild type zinc fingers which bind defg and abcd, respectively). Finger X would have a mutated polypeptide sequence. Preferably, finger X would have mutations at one or more of the base-contacting positions, i.e., finger X would have the same polypeptide sequence as a wild type zinc finger except that at least one of the four amino residues at the primary positions would differ. Similarly, to design a three-finger zinc protein that would bind to a 10 base sequence abcXXXXhij (wherein each “X” is base that would be specified in a particular application), fingers X and Z have the same sequence as the wild type zinc fingers which bind ghij and abcd, respectively, while finger Y would have residues at one or more base-coating positions which differ from those in a wild type finger. The present method can employ multi-fingered proteins in which more than one finger differs from a wild type zinc finger. The present method can also employ multi-fingered protein in which the amino acid sequence in all the fingers have been changed, including those designed by combinatorial chemistry or other protein design and binding assays.

It is also possible to design or select a zinc finger protein to bind to a targeted polynucleotide in which more than four bases have been altered. In this case, more than one finger of the binding protein must be altered. For example, in the 10 base sequence XXXdefgXXX, a three-finger binding protein could be designed in which fingers X and Z differ from the corresponding fingers in a wild type zinc finger, while finger Y will have the same polypeptide sequence as the corresponding finger in the wild type fingers which binds to the subsite defg. Binding proteins having more than three fingers can also be designed for base sequences of longer length. For example, a four finger-protein will optimally bind to a 13 base sequence, while a five-finger protein will optimally bind to a 16 base sequence. A multi-finger protein can also be designed in which some of the fingers are not involved in binding to the selected DNA. Slight variations are also possible in the spacing of the fingers and framework.

Methods for designing and identifying a zinc finger protein with desired nucleic acid binding characteristics also include those described in WO98/53060, which reports a method for preparing a nucleic acid binding protein of the Cys2-His2 zinc finger class capable of binding to a nucleic acid quadruplet in a target nucleic acid sequence. Exemplary methods for identifying zinc finger proteins that activate or repress a gene are known in the art (e.g., Yokoi et al., Molecular Therapy 15(10:1917-23, 2007; Zhang et al., Molecular Therapy 13:S305, 2006; Lai and Elliot on-line publication at criticalimprov.com/index.php/surg/article/view/294/444).

Zinc finger proteins useful in the present method can comprise at least one zinc finger polypeptide linked via a linker, preferably a flexible linker, to at least a second DNA binding domain, which optionally is a second zinc finger polypeptide. The zinc finger protein may contain more than two DNA-binding domains, as well as one or more effector domains. The zinc finger polypeptides used in the present method can be engineered to recognize a selected target site in the gene of choice, for example the SERCA2 gene. Typically, a backbone from any suitable C2H2-ZFP, such as SPA, SPIC, or ZIF268, is used as the scaffold for the engineered zinc finger polypeptides (see, e.g., Jacobs, EMBO J. (1992) 11:4507; and Desjarlais & Berg, Proc. Natl. Acad. Sci. USA (1993) 90:2256-2260). A number of methods can then be used to design and select a zinc finger polypeptide with high affinity for its target. A zinc finger polypeptide can be designed or selected to bind to any suitable target site in the target gene, with high affinity.

Any suitable method known in the art can be used to design and construct nucleic acids encoding zinc finger polypeptides, e.g., phage display, random mutagenesis, combinatorial libraries, computer/rational design, affinity selection, PCR, cloning from cDNA or genomic libraries, synthetic construction and the like. (see, e.g., U.S. Pat. No. 5,786,538; Wu et al., Proc. Natl. Acad. Sci. USA (1995) 92:344-348; Jamieson et al., Biochemistl. (1994) 33:5689-5695; Rebar & Pabo, Science (1994) 263:671-673; Choo & Klug, Proc. Natl. Acad. Sci. USA (1994) 91: 11168-11172; Pomerantz et al., Science, 267:93-96 (1995); Pomerantz et al., Proc. Natl. Acad. Sci. USA (1995) 92:9752-9756; Liu et al., Proc. Natl. Acad. Sci. USA (1997) 94:5525-5530; and Desjarlais & Berg, (1994) Proc. Natl. Acad. Sci. USA 91:11-99-11103).

Zinc finger proteins useful in the method can be made by any recombinant DNA technology method for gene construction. For example, PCR based construction can be used. Ligation of desired fragments can also be performed, using linkers or appropriately complementary restriction sites. One can also synthesize entire finger domain or parts thereof by any protein synthesis method. Preferred for cost and flexibility is the use of PCR primers that encode a finger sequence or part thereof with known base pair specificity, and that can be reused or recombined to create new combinations of fingers and ZFP sequences.

The amino acid linker should be flexible, a beta turn structure is preferred, to allow each three finger domain to independently bind to its target sequence and avoid steric hindrance of each other's binding. Linkers can be designed and empirically tested.

If a recognition code is incomplete, or if desired, in one embodiment, the ZFP can be designed to bind to non-contiguous target sequences. For example, a target sequence for a six-finger ZFP can be a nine base pair sequence (recognized by three fingers) with intervening bases (that do not contact the zinc finger nucleic acid binding domain) between a second nine base pair sequence (recognized by a second set of three fingers). The number of intervening bases can vary, such that one can compensate for this intervening distance with an appropriately designed amino acid linker between the two three-finger parts of ZFP. A range of intervening nucleic acid bases in a target binding site is preferably 20 or less bases, more preferably 10 or less, and even more preferably 6 or less bases. It is of course recognized that the linker must maintain the reading frame between the linked parts of ZFP protein.

A minimum length of a linker is the length that would allow the two zinc finger domains to be connected without providing steric hindrance to the domains or the linker. A linker that provides more than the minimum length is a “flexible linker.” Determining the length of minimum linkers and flexible linkers can be performed using physical or computer models of DNA-binding proteins bound to their respective target sites as are known in the art.

The six-finger zinc finger peptides can use a conventional “TGEKP” linker to connect two three-finger zinc finger peptides or to add additional fingers to a three-finger protein. Other zinc finger peptide linkers, both natural and synthetic, are also suitable.

A useful zinc finger framework is that of ZIF268 (see WO00/23464 and references cited therein.), however, others are suitable. Examples of known zinc finger nucleotide binding polypeptides that can be truncated, expanded, and/or mutagenized in order to change the function of a nucleotide sequence containing a zinc finger nucleotide binding motif includes TFIIIA and zif268. Other zinc finger nucleotide binding proteins are known to those of skill in the art. The murine CYS2-HiS2 zinc finger protein Zif268 is structurally well characterized of the zinc finger proteins (Pavletich and Pabo, Science (1991) 252:809-817; Elrod-Erickson et al., Structure (London) (1996) 4:1171-1180; and Swirnoff et al., Mol. Cell. Biol. (1995) 15:2275-2287). DNA recognition in each of the three zinc finger domains of this protein is mediated by residues in the N-terminus of the alpha-helix contacting primarily three nucleotides on a single strand of the DNA. The operator binding site for this three finger protein is 5′-GCGTGGGCG-'3. Structural studies of Zif268 and other related zinc finger-DNA complexes (Elrod-Erickson et al., Structure (London) (1998) 6:451-464; Kim and Berg, Nature Structural Biology (1996) 3:940-945; Pavletich and Pabo, Science (1993) 261:1701-1707; Houbaviy et al., Proc. Natl. Acad. Sci. USA (1996) 93:13577-13582; Fairall et al., Nature (London) (1993) 366:483-487; Wuttke et al., J. Mol. Biol. (1997) 273:183-206; Nolte et al., Proc. Natl. Acad. Sci. USA (1998) 95:2938-2943; and Narayan et al., J. Biol. Chem. (1997) 272:7801-7809) have shown that residues from primarily three positions on the α-helix, −1, 3, and 6, are involved in specific base contacts. Typically, the residue at position −1 of the α-helix contacts the 3′ base of that finger's subsite while positions 3 and 6 contact the middle base and the 5′ base, respectively.

However, it should be noted that at least in some cases, zinc finger domains appear to specify overlapping 4 bp sites rather than individual 3 bp sites. In Zif268, residues in addition to those found at helix positions −1, 3, and 6 are involved in contacting DNA (Elrod-Erickson et al., Structure (1996) 4:1171-1180). Specifically, an aspartate in helix position 2 of the middle finger plays several roles in recognition and makes a variety of contacts. The carboxylate of the aspartate side chain hydrogen bonds with arginine at position −1, stabilizing its interaction with the 3′-guanine of its target site. This aspartate may also participate in water-mediated contacts with the guanine's complementary cytosine. In addition, this carboxylate is observed to make a direct contact to the N4 of the cytosine base on the opposite strand of the 5′-guanine base of the finger 1 binding site. It is this interaction which is the chemical basis for target site overlap.

Any suitable method of protein purification known to those of skill in the art can be used to purify the zinc finger proteins of the invention (see Sambrook et al., Molecular Cloning: A Laboratory Manual (2d ed., Cold Spring Harbor Laboratory Press, Plainview, N.Y.) (1989)). In addition, any suitable host can be used, e.g., bacterial cells, insect cells, yeast cells, mammalian cells, and the like.

Although in some cases, a zinc finger protein itself is sufficient for modulating gene expression, the zinc finger protein is preferably fused to an effector domain (or regulatory domain or functional domain), i.e., a protein domain which activates or represses gene expression, such as a transcription factor or transcription activation domain thereof. Examples of regulatory domains include proteins or effector domains of proteins such as transcription factors and co-factors, e.g., KRAB, MAD, ERD, SID, nuclear factor kappa B subunit p65, early growth response factor 1, and nuclear hormone receptors, VP16 and VP64, endonucleases, integrases, recombinases, methyltransferases, histone acetyltransferases, histone deacetylases, mutases, restriction enzymes, etc. In particular embodiments, the transcription factor or transcription activation domain thereof is selected from the group consisting of p53, NFAT, NF-κB and VP16.

Transcription factor polypeptides from which one can obtain a regulatory domain include those that are involved in regulated and basal transcription. Such polypeptides include transcription factors, their effector domains, coactivators, silencers, nuclear hormone receptors (see, e.g., Goodrich et al., Cell (1996) 84:825-830) for a review of proteins and nucleic acid elements involved in transcription. Transcription factors in general are reviewed in Barnes and Adcock, Clin. Exp. Allerg v 25 Suppl. 2:46-49 (1995) and Roeder, Methods Enz. (1996) 273:165-171. Databases dedicated to transcription factors are also known (see, e.g., Williams, Science (1995) 269:630). Nuclear hormone receptor transcription factors are described in, for example, Rosen et al., J. Med. Chem. (1995) 38:4855-4874. The C/EBP family of transcription factors are reviewed in Wedel et al., Immunobiology (1995) 193:171-185. Coactivators and co-repressors that mediate transcription regulation by nuclear hormone receptors are reviewed in, for example, Meier, Eur. J. Endocrinol. (1996) 134(2):158-9; Kaiser et al., Trends Biochem. Sci. (1996) 21:342-345; and Utley et al., Nature (1998) 394:498-502. GATA transcription factors, which are involved in regulation of hematopoiesis, are described in, for example, Simon, Nat. Genet. (1995) 11:9-11; and Weiss et al., Exp. Hematol. 23:99-107. TATA box binding protein (T13P) and its associated TAF polypeptides (which include TAF30, TAF55, TAF80, TAF110, TAF150, and TAF250) are described in Goodrich & Tjian, Curr. Opin. Cell Biol. (1994) 6:403-409 and Hurley, Curr. Opin. Struct. Biol. (1996) 6:69-75. The STAT family of transcription factors are reviewed in, for example, Barahmand-Pour et al., Curr. Top. Microbiol. Immunol. (1996) 211:121-128. Transcription factors involved in disease are reviewed in Aso et al., J. Clin. Invest. (1996) 97:1561-1569.

As used herein, “specifically binds to a target nucleotide sequence” means that the binding affinity of a zinc finger protein to a specified target nucleotide sequence of a nucleic acid is statistically higher than the binding affinity of the same zinc finger protein to a generally comparable, but non-target nucleotide sequence, e.g., a GNN sequence without matching code sequence for the particular zinc finger protein. Normally, the binding affinity of a zinc finger protein to a specified target nucleic acid sequence is at least 1.5 fold, and preferably 2 fold or 5 fold, of the binding affinity of the same zinc finger protein to a non-target nucleic acid sequence. It also refers to binding of a zinc-finger-protein-nucleic-acid-binding domain to a specified nucleic acid target sequence to a detectably greater degree, e.g., at least 1.5-fold over background, than its binding to non-target nucleic acid sequences and to the substantial exclusion of non-target nucleic acids. The zinc finger protein's Kd to each nucleotide sequence can be compared to assess the binding specificity of the zinc finger protein to a particular target nucleotide sequence.

As used herein, the term “target site” or “target nucleotide sequence” is the nucleic acid sequence recognized by a ZFP. A single target site typically has about four to about ten base pairs. Typically, a two-fingered ZFP recognizes a four to seven base pair target site, a three-fingered ZFP recognizes a six to ten base pair target site, and a six fingered ZFP recognizes two adjacent nine to ten base pair target sites.

As used herein, a “target nucleotide sequence within a target gene” refers to a functional relationship between the target nucleotide sequence and the target gene in that binding of a zinc-finger-protein to the target nucleotide sequence will modulate the expression of the target gene. The target nucleotide sequence can be physically located anywhere inside the boundaries of the target gene, e.g., 5′ ends, coding region, 3′ ends, upstream and downstream regions outside of cDNA encoded region, or inside enhancer or other regulatory region, and can be proximal or distal to the target gene. The target nucleotide sequence is any location within the target gene whose expression is to be regulated which provides a suitable location for controlling expression. The target nucleotide sequence may be within the coding region or upstream or downstream thereof. For activation, upstream from ATG translation start codon is preferred, most preferably upstream of TATTA box but not exceeding 1000 bp from the start of transcription. For repression, upstream from the ATG translation start codon is also preferred, but preferably downstream from TATTA box.

In some embodiments of the present invention the target gene is a Sarco/Endoplasmic Reticulum Ca²⁺-ATPase (SERCA) gene. In one aspect, the target gene is SERCA2a. In certain embodiments, the SERCA2a protein is expressed from an endogenous SERCA2a gene.

SERCA proteins reside in the sarcoplasmic reticulum (SR) within muscle cells. Contraction of the cardiac muscle is reported to be dependent on mobilization Ca²⁺ from intracellular stores or the SR. SERCA is a Ca²⁺ ATPase which transfers Ca²⁺ from the cytosol of the cell to the lumen of the sarcoplasmic reticulum (SR) at the expense of ATP hydrolysis. SERCA proteins are encoded by three genes (SERCA1, 2 and 3) located on separate chromosomes. SERCA transcripts are expressed and alternatively spliced in a tissue-dependent manner. The resulting mRNA species encode different SERCA protein isoforms and differ in 3′-untranslated regions (UTR). SERCA protein isoforms differ in their Ca²⁺ affinity, resistance to oxidative stress and modulation by sarcolipin, phospholamban (PLB/PLN), and Ca²⁺/calmodulin kinase II. The PLB:SERCA ratio has been shown to significantly modulate smooth muscle Ca²⁺ concentrations.

In the heart, PLB functions as an inhibitor of SERCA by decreasing the affinity for Ca²⁺ and phosphorylation of the PLB relieves the inhibition. Hence, the rate at which SERCA moves Ca²⁺ across the SR membrane can be controlled by phospholamban (PLB/PLN) under β-adrenergic stimulation. When PLB is associated with SERCA, the rate of Ca²⁺ movement is reduced, upon dissociation of PLB, Ca²⁺ movement increases. For example, SERCA2 actively transports about 70 to 80% of free calcium ions into the SR intracellular space during diastolic relaxation of cardiac muscle.

The nucleotide sequence of SERCA2 and its isoforms has been identified and sequenced from various mammalian species and is about 90%+conserved among mammalian species. For example, SERCA2 genes have been identified including human GenBank sequences NM_{—}170665 (Lytton and MacLennan, J Biol Chem (1998) 263(29):15024-15031); NM_{—}001681 (Id.); NM_{—}006241 (Park et al., J Biol Chem (1994) 269(2):944-954); NM 001003214 (Autry and Jones, J Biol Chem (1997) 272(25):15872-15880); BCO35588 (Strausberg et al., Proc Natl Mad Sci USA (2002) 99(26):16899-16903); AY186578 (Gelebart et al., Biochem Biophys Res Comm (2003) 303(2):676-684); and mouse GenBank sequences NM_{—}026482 (Du et al., Arch Biochem Biophys (1995) 316(1):302-310); NM_{—}213616 (Hunter et al., Genomics (1993) 18(3):510-519); NM_{—}009722 (Hsu et al., Biochem Biophys Res Comm (1993) 197(3):1483-1491); AJ131870 (Ver Heyen et al., Mamm Genome (2000) 11(2):159-163); BCO54531 (Strausberg et al., Proc Natl Acad Sci USA (2002) 99(26):16899-16903); BCO54748 (Id.); AJ131821 (Ver Heyen et al., Mamm Genome (2000) 11(2):159-163); AJ223584 (Id.); AF029982 (Id.); and AF039893 (Schoenfeld and Lowe, direct submission (18-Dec.-1997), Cardiovascular Research, Genentech, 1 DNA Way, South San Francisco, Calif. 94080, USA). All of the above sequences are publicly available.

In some embodiments of the methods of the invention, an polynucleotide encoding SERCA2a is co-administered with a ZFP that induces SERCA2a expression, a ZFP effector domain fusion protein, or a polynucleotide encoding the ZFP protein or ZFP effector domain fusion. The SERCA2 polynucleotide to be used in the invention may be DNA or RNA, but will in some embodiments be a complementary DNA (cDNA) sequence. The polynucleotide sequences used in the invention must be (a) expressible and (b) either non-replicating or engineered by means well known in the art so as not to replicate into the host genome. In certain embodiments, a polynucleotide which operatively encodes a SERCA2 protein will be used in the invention as part of a recombinant expression vector, most preferably an adenovirus construct. In some aspects, the polynucleotide encoding the ZFP or ZFP fusion protein and the SERCA2a gene are contained within the same expression vector.

Alternatively, the nucleic acid molecule encoding the ZFP that induces SERCA2a expression or ZFP fusion protein and the SERCA2a gene may be contained within the separate expression vectors. In one aspect, such separate expression vectors are administered simultaneously. In another aspect, the expression vectors are administered separately with the ZFP or ZFP effector domain fusion protein encoding expression vector administered first and the SERCA2a encoding expression vector administered second. In another aspect, the expression vectors are administered separately with the SERCA2a encoding expression vector administered first and the ZFP or ZFP effector domain fusion protein encoding expression vector administered second.

Information regarding the genomic structure of the human SERCA2 gene can be found on the NCBI website for GeneID: 488 (termed ATP2A2). The hSERCA2 gene is located on chromosome 12 position q24.1 in Contig NT_{—}009770.8, spans 70 kb, and is organized into 21 exons with 20 intervening introns. The last two exons of the pre-mRNA produce the cardiac/slow-twitch muscle-specific SERCA2a isoform and the ubiquitous SERCA2b isoform by alternatively splicing. In addition, 2.4 kb of the 5′-regulatory region the human SERCA2 gene has been cloned and characterized (Zarain-Herzberg and Alvarez-Fernandez, ScientificWorldJournal 2:1469-83, 2002). The sequence of the proximal 225-bp regulatory region of the SERCA2 genes is 80% GC-rich and is conserved among human, rabbit, rat, and mouse species. It contains a TATA-like-box, an E-box/USF sequence, a CAAT-box, four Sp1 binding sites, and a thyroid hormone responsive element (TRE). There are two other conserved regulatory regions located between positions −410 to −−661 bp and from −919 to −1410 bp. Among the DNA cis-elements present in these two regulatory regions there are potential binding sites for: GATA-4, -5, -6, Nkx-2.5/Csx, OTF-1, USF, MEF-2, SRF, PPAR/RXR, AP-2, and TREs. In addition, the human gene has several repeated sequences mainly of the Alu and L2 type located upstream from position −1.7 kb, spanning in a continuous fashion for more than 40 kb. In this study, we report the cloning of 2.4 kb of 5-regulatory region and demonstrate that the proximal promoter region is sufficient for expression in cardiac myocytes, and the region from −225 to −1232 bp contains regulatory DNA elements which down-regulate the expression of the SERCA2 gene in neonatal cardiomyocytes. (Zarain-Herzberg and Alvarez-Fernandez, ScientificWorldJournal 2:1469-83, 2002). In some embodiments, one or more of the regulatory regions or binding sites may be targeted for binding with a zinc finger protein to modulate expression of the SERCA2 gene.

The term “operatively linked” or “operatively associated” means that two or more molecules are positioned with respect to each other such that they act as a single unit and effect a function attributable to one or both molecules or a combination thereof. For example, a polynucleotide sequence encoding a zinc finger protein can be operatively linked to a regulatory element, in which case the regulatory element confers its regulatory effect on the polynucleotide similarly to the way in which the regulatory element would affect a polynucleotide sequence with which it normally is associated with in a cell. A first polynucleotide coding sequence also can be operatively linked to a second (or more) coding sequence such that a chimeric polypeptide can be expressed from the operatively linked coding sequences. The chimeric polypeptide can be a fusion polypeptide, in which the two (or more) encoded peptides are translated into a single polypeptide, i.e., are covalently bound through a peptide bond; or can be translated as two discrete peptides that, upon translation, can operatively associate with each other to form a stable complex. In one example, a polynucleotide encoding a zinc finger protein that specifically binds to a target nucleotide sequence within the promoter region of the SERCA2a gene is operatively linked to a polynucleotide encoding an effector domain, resulting in a fused polypeptide of the zinc finger protein and the effector domain.

The term “promoter,” as used herein, refers to a region of sequence determinants located upstream from the start of transcription of a gene and which are involved in recognition and binding of RNA polymerase and other proteins to initiate and modulate transcription. A basal promoter is the minimal sequence necessary for assembly of a transcription complex required for transcription initiation. Basal promoters frequently include a “TATA box” element usually located between 15 and 35 nucleotides upstream from the site of initiation of transcription. Basal promoters also sometimes include a “C CAAT box” element (typically a sequence CCAAT) and/or a GGGCG sequence, usually located between 40 and 200 nucleotides, preferably 60 to 120 nucleotides, upstream from the start site of transcription. “Constitutive” promoters actively promote transcription under most, but not necessarily all, environmental conditions and states of development or cell differentiation.

As used herein, “expression cassette” refers to a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The zinc finger-effector fusions of the present methods may be chimeric. The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In some embodiments of the invention, a nucleic acid molecule encoding the zinc finger protein is contained within an expression cassette.

As used herein, “significant increase” refers to an increase in gene expression, biological activity that is larger than the margin of error inherent in the measurement technique, preferably an increase by about 2-fold or greater of the activity without the ZFP, more preferably an increase by about 5-fold or greater, and most preferably an increase by about 10-fold or greater.

As used herein, “activator protein” refer to a protein that binds to operator of DNA or to RNA to enhance transcription or translation, respectively.

As used herein, “activation” refer to enhancement of transcription or translation by binding of activator protein to specific site on DNA or mRNA. Preferably, activation includes a significant change in transcription or translation level of at least 1.5 fold, more preferably at least two fold, and even more preferably at least five fold.

In some embodiments, the zinc finger protein is administered as a nucleic acid encoding the zinc finger protein. One aspect of the present invention contemplates transfer of such nucleic acids to a cardiac myocyte using viral or non-viral methods of gene transfer. In one embodiment, a polynucleotide encoding a zinc finger protein is incorporated into a viral vector to mediate transfer to a cell. Additional expression constructs encoding other therapeutic agents as described herein may also be transferred via viral transduction using infectious viral particles, for example, by transformation with an adeno-associated virus (AAV). Alternatively, a retrovirus, bovine papilloma virus, an adenovirus vector, a lentiviral vector, a vaccinia virus, a polyoma virus, or an infective virus that has been engineered to express may be used. Similarly, nonviral methods which include, but are not limited to, direct delivery of DNA such as by perfusion, naked DNA transfection, liposome mediated transfection, encapsulation, and receptor-mediated endocytosis may be employed. These techniques are well known to those of skill in the art, and the particulars thereof do not lie at the crux of the present invention and thus need not be exhaustively detailed herein. For example, a viral vector is used for the transduction of cardiac cells to deliver a therapeutically significant polynucleotide to a cell. The virus may gain access to the interior of the cell by a specific means such as receptor-mediated endocytosis, or by non-specific means such as pinocytosis.

Adeno-associated virus (AAV) has shown promise for delivering genes for gene therapy in clinical trials in humans. As the only viral vector system based on a nonpathogenic and replication-defective virus, recombinant AAV virions have been successfully used to establish efficient and sustained gene transfer of both proliferating and terminally differentiated cells in a variety of tissues.

The AAV genome is a linear, single-stranded DNA molecule containing about 4681 nucleotides. The AAV genome generally comprises an internal nonrepeating genome flanked on each end by inverted terminal repeats (ITRs). The ITRs are approximately 145 base pairs (bp) in length. The ITRs have multiple functions, including as origins of DNA replication, and as packaging signals for the viral genome. The internal nonrepeated portion of the genome includes two large open reading frames, known as the AAV replication (rep) and capsid (cap) genes. The rep and cap genes code for viral proteins that allow the virus to replicate and package into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP 1, VP2, and VP3.

AAV has been engineered to deliver genes of interest by deleting the internal nonrepeating portion of the AAV genome (i.e., the rep and cap genes) and inserting a heterologous gene between the ITRs. The heterologous gene is typically functionally or operatively linked to a heterologous promoter (constitutive, cell-specific, or inducible) capable of driving gene expression in the patient's target cells under appropriate conditions. Termination signals, such as polyadenylation sites, can also be included.

The term “AAV vector” means a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Despite the high degree of homology, the different serotypes have tropisms for different tissues. The receptor for AAV1 is unknown; however, AAV1 is known to transduce skeletal and cardiac muscle more efficiently than AAV2. Since most of the studies have been done with pseudotyped vectors in which the vector DNA flanked with AAV2 ITR is packaged into capsids of alternate serotypes, it is clear that the biological differences are related to the capsid rather than to the genomes. Recent evidence indicates that DNA expression cassettes packaged in AAV 1 capsids are at least 1 log 10 more efficient at transducing cardiomyocytes than those packaged in AAV2 capsids. In one embodiment, the viral delivery system is an adeno-associated viral delivery system. The adeno-associated virus can be of serotype I (AAV 1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9).

Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, as long as the sequences provide for functional rescue, replication and packaging.

AAV vectors must have one copy of the AAV inverted terminal repeat sequences (ITRs) at each end of the genome in order to be replicated, packaged into AAV particles and integrated efficiently into cell chromosomes. However, the nucleic acid promoted by ITR can be any desired sequence. In one embodiment, the nucleic acid encodes a zinc finger protein capable of inducing expression of SERCA2a, or a nucleic acid encoding a zinc finger protein that binds to the promoter region of the SERCA2a gene, and fused to an effector domain, wherein the encoded protein increases expression of SERCA2a.

The ITR consists of nucleotides 1 to 145 at the left end of the AAV DNA genome and the corresponding nucleotides 4681 to 4536 (i.e., the same sequence) at the right hand end of the AAV DNA genome. Thus, AAV vectors must have a total of at least 300 nucleotides of the terminal sequence. So, for packaging large coding regions into AAV vector particles, it is important to develop the smallest possible regulatory sequences, such as transcription promoters and polyA addition signal. In this system, the adeno-associated viral vector comprising the inverted terminal repeat (ITR) sequences of adeno-associated virus and a nucleic acid encoding a zinc finger protein or zinc finger protein fused to an effector domain, wherein the inverted terminal repeat sequences promote expression of the nucleic acid in the absence of another promoter.

Accordingly, as used herein, AAV means all serotypes of AAV. Thus, it is routine in the art to use the ITR sequences from other serotypes of AAV since the ITRs of all AAV serotypes are expected to have similar structures and functions with regard to replication, integration, excision and transcriptional mechanisms.

AAV is also a helper-dependent virus. That is, it requires coinfection with a helper virus (e.g., adenovirus, herpesvirus or vaccinia), in order to form AAV virions. In the absence of coinfection with a helper virus, AAV establishes a latent state in which the viral genome inserts into a host cell chromosome, but infectious virions are not produced. Subsequent infection by a helper virus “rescues” the integrated genome, allowing it to replicate and package its genome into an infectious AAV virion. While AAV can infect cells from different species, the helper virus must be of the same species as the host cell. Thus, for example, human AAV will replicate in canine cells coinfected with a canine adenovirus.

The term “AAV helper functions” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

Accordingly, the term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs and vectors that encode Rep and/or Cap expression products have been described.

Typically, recombinant AAV (rAAV) virus is made by cotransfecting a plasmid containing the gene of interest flanked by the two AAV terminal repeats and/or an expression plasmid containing the wild-type AAV coding sequences without the terminal repeats, for example pIM45. The cells are also infected and/or transfected with adenovirus and/or plasmids carrying the adenovirus genes required for AAV helper function. rAAV virus stocks made in such fashion are contaminated with adenovirus which must be physically separated from the rAAV particles (for example, by cesium chloride density centrifugation or column chromatography). Alternatively, adenovirus vectors containing the AAV coding regions and/or cell lines containing the AAV coding regions and/or some or all of the adenovirus helper genes could be used. Cell lines carrying the rAAV DNA as an integrated provirus can also be used.

The term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

Accordingly, “accessory function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid.

In particular, it has been demonstrated that the full-complement of adenovirus genes is not required for accessory helper functions. In particular, adenovirus mutants incapable of DNA replication and late gene synthesis have been shown to be permissive for AAV replication. Similarly, mutants within the E2B and E3 regions have been shown to support AAV replication, indicating that the E2B and E3 regions are probably not involved in providing accessory functions. However, adenoviruses defective in the E1 region, or having a deleted E4 region, are unable to support AAV replication. Thus, E1A and E4 regions are likely required for AAV replication, either directly or indirectly. Other characterized Ad mutants include: E1B; E2A; E2B; E3; and E4. Although studies of the accessory functions provided by adenoviruses having mutations in the E1B coding region have produced conflicting results, recently it has been reported that E1B55k is required for AAV virion production, while E1B19k is not.

Exemplary accessory function vectors comprise an adenovirus VA RNA coding region, an adenovirus E4 ORF6 coding region, an adenovirus E2A 72 kD coding region, an adenovirus E1A coding region, and an adenovirus EIB region lacking an intact E1B55k coding region.

By “capable of supporting efficient rAAV virion production” is meant the ability of an accessory function vector or system to provide accessory functions that are sufficient to complement rAAV virion production in a particular host cell at a level substantially equivalent to or greater than that which could be obtained upon infection of the host cell with an adenovirus helper virus. Thus, the ability of an accessory function vector or system to support efficient rAAV virion production can be determined by comparing rAAV virion titers obtained using the accessory vector or system with titers obtained using infection with an infectious adenovirus. More particularly, an accessory function vector or system supports efficient rAAV virion production substantially equivalent to, or greater than, that obtained using an infectious adenovirus when the amount of virions obtained from an equivalent number of host cells is not more than about 200 fold less than the amount obtained using adenovirus infection, more preferably not more than about 100 fold less, and most preferably equal to, or greater than, the amount obtained using adenovirus infection.

Hence, by “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

Similarly, a “recombinant AAV virion,” or “rAAV virion” is defined herein as an infectious, replication-defective virus including an AAV protein shell, encapsidating a heterologous nucleotide sequence of interest which is flanked on both sides by AAV ITRs. A rAAV virion is produced in a suitable host cell which has had an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

The AAV system of the invention, may also include a sequence encoding a selectable marker. The phrase, “selectable marker” or “selectable gene product” as used herein, refers to the use of a gene which may include but is not limited to: bacterial aminoglycoside 3′ phosphotransferase gene (also referred to as the neo gene) which confers resistance to the drug G418 in mammalian cells; bacterial hygromycin G phosphotransferase (hyg) gene which confers resistance to the antibiotic hygromycin; and the bacterial xanthine-guanine phosphoribosyl transferase gene (also referred to as the gpt gene) which confers the ability to grow in the presence of mycophenolic acid. In addition, the AAV system of the invention may also include sequences encoding a visual detectable marker, e.g., green fluorescent protein (GFP) or any other detectable marker standard in the art and can be identified and utilized by one skilled in the art without undue experimentation.

Stable expression of a transgene using an AAV vector has been demonstrated in the art. While not wishing to be bound to any particular theory, it is believed that a number of steps need to occur in order to obtain stable expression from AAV vectors such as MYDICARE®: (1) vector receptor binding on the target cell, (2) vector entry, (3) single-stranded DNA conversion to “hair-pinned” double-stranded DNA (dsDNA) requiring new DNA synthesis, (4) hair-pinned dsDNA conversion to covalently-closed circular DNA intermediates, and (5) circular dsDNA intermediate conversion into high-molecular weight circular concatemers. Studies suggest that sustained stable expression from AAV vectors results primarily from stable circular extrachromosomal concatamers (Yon et al., J Virol 79:364-379, 2005; and Schnepp et al., J Virol 79:14793-14803. 2005). Molecular studies suggest that initial transgene expression from single-stranded AAV vectors carrying transgenes of similar length as the SERCA2a cDNA may be due to the generation of unstable, short-lived non-circular dsDNA intermediates formed from the annealing of vector DNA genomes of opposite polarity followed by gradually increasing stable expression from circular, stable vector dsDNA genomes over time (Wang, et al., PNAS. 2007; 104:13104-13109). The expected kinetics of expression of MYDICAR® were based on data utilizing a GFP reporter gene with the same molecular vector backbone as MYDICAR®. However, this transgene is much shorter than SERCA2a, and based on molecular studies, the shorter length transgenes can lead to generation of vector particles pre-packaged with dsDNA (i.e., obviating the need for step 3 above in the pathway). It is known that the initial transduction efficiency as measured by transgene expression is higher from dsDNA vs. ssDNA AAV vectors, potentially explaining the differences in initial transduction frequency from vectors carrying transgenes of varying lengths, even though no other differences in the molecular backbone exist.

In some embodiments, the AAV vector is a double-stranded vector. Double-stranded AAV (ds AAV) vectors are known in the art. In one report, one of the AAV inverted terminal repeats (ITRs) was mutated, leading to nearly exclusive packaging of hairpin-like, ds AAV DNA genomes (Wang et al., Gene Therapy 10:2105-11, 2003). Examination of this ds AAV vector in vitro and in vivo and molecular characterization of the vector DNA demonstrated enhanced gene transfer of the ds AAV vector as compared to the conventional ssAAV vector. This exemplary ds AAV vector was constructed by deleting the D-sequence of the 50 ITR with MscI digestion. MscI removed the D-sequence and the terminal resolution site (trs) (nucleotides 122-144 of AAV2 genome, GenBank #NC_{—}001401). The ITR on the 30 terminus of the vector remained intact (i.e., wild type) (Wang et al., Gene Therapy 10:2105-11, 2003).

Some skilled in the art have circumvented some of the limitations of adenovirus-based vectors by using adenovirus “hybrid” viruses, which incorporate desirable features from adenovirus as well as from other types of viruses as a means of generating unique vectors with highly specialized properties. For example, viral vector chimeras were generated between adenovirus and adeno-associated virus (AAV). These aspects of the invention do not deviate from the scope of the invention described herein.

Another method for delivery of the polynucleotide for gene therapy involves the use of an adenovirus expression vector. “Adenovirus expression vector” is meant to include those constructs containing adenovirus sequences sufficient (a) to support packaging of the construct and/or (b) to ultimately express a tissue and/or cell-specific construct that has been cloned therein.

In one form of the invention, the expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, Seminar in Virology, 1992; 3:237-252). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus growth and manipulation is known to those of skill in the art, and exhibits broad host range in vitro and in vivo. This group of viruses can be obtained in high titers, e.g., 109 to 1011 plaque-forming units per mL, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and/or therapeutic potential as in vivo gene transfer vectors.

Adenovirus vectors have been used in eukaryotic gene expression and vaccine development. Recently, animal studies suggested that recombinant adenovirus could be used for gene therapy (see, e.g., Stratford-Perricaudet et al., Hum. Gene. Ther., 1991; 1:242-256; Rich et al., 1993). Studies in administering recombinant adenovirus to different tissues include muscle injection, peripheral intravenous injections and stereotactic inoculation into the brain. Recombinant adenovirus and adeno-associated virus can both infect and transduce non-dividing human primary cells.

While the use of adenovirus vectors is contemplated, such use in cardiovascular gene therapy trials is currently limited by short-lived transgene expression. (Vassalli G, et al., Int. J. Cardiol., 2003; 90 (2-3):229-38). This is due to cellular immunity against adenoviral antigens. Improved “gutless” adenoviral vectors have reduced immunogenicity, yet still are ineffective if maximal expression of the transgene for more than six months is needed or desired for therapeutic effect (Gilbert R, et al., Hum. Mol. Genet., 2003; 12(11):1287-99). AAV vectors have demonstrated long term expression (>1 year) and are the preferred vector for therapeutic effects where expression is needed long-term (Daly T M, et al., Gene Ther., 2001; 8(17):1291-8).

Nucleic acids encoding the zinc finger proteins of the invention may be delivered to cardiac muscle by methods known in the art (see e.g., US Patent Appln. Publication No. US 2009/0209631). For example, cardiac cells of a large mammal may be transfected by a method that includes dilating a blood vessel of the coronary circulation by administering a vasodilating substance to said mammal prior to, and/or concurrent with, administering the nucleic acids. In some embodiments, the method includes administering the nucleic acids into a blood vessel of the coronary circulation in vivo, wherein nucleic acids are infused into the blood vessel over a period of at least about three minutes, wherein the coronary circulation is not isolated or substantially isolated from the systemic circulation of the mammal, and wherein the nucleic acids transfect cardiac cells of the mammal.

In some embodiments, the subject can be a human, an experimental animal, e.g., a rat or a mouse, a domestic animal, e.g., a dog, cow, sheep, pig or horse, or a non-human primate, e.g., a monkey. The subject may be suffering from a cardiac disorder, such as heart failure, ischemia, myocardial infarction, congestive heart failure, arrhythmia, transplant rejection and the like. In a preferred embodiment, the subject is suffering from heart failure. In another particular embodiment, the subject is suffering from arrhythmia. As used herein, the term “misexpression” refers to a non-wild type pattern of gene expression. It includes: expression at non-wild type levels, i.e., over- or underexpression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus. In one embodiment, the subject is a human. For example, the subject is between ages 18 and 65. In another embodiment, the subject is a non-human animal.

In one embodiment, the subject has or is at risk for heart failure, e.g. a non-ischemic cardiomyopathy, mitral valve regurgitation, ischemic cardiomyopathy, or aortic stenosis or regurgitation. In one embodiment, the subject is in need of improved sarcoplasmic reticulum Ca²⁺ uptake in the cardiac muscle.

In some embodiments, transfection of cardiac cells with nucleic acid molecules encoding a zinc finger protein or zinc finger protein fused to an effector domain increases lateral ventricle fractional shortening. In some embodiments, the mammal is human and the disease is congestive heart failure. In some embodiments, the transfection of the cardiac cells increases lateral ventricle fractional shortening when measured about 4 months after said infusion by at least 25% as compared to lateral ventricle fractional shortening before infusion of the polynucleotide. In some embodiments, the transfection of the cardiac cells results in an improvement in a measure of cardiac function selected from the group consisting of expression of SERCA2a protein, fractional shortening, ejection fraction, cardiac output, time constant of ventricular relaxation, and regurgitant volume.

A treatment can be evaluated by assessing the effect of the treatment on a parameter related to contractility. For example, SR Ca²⁺ATPase activity or intracellular Ca²⁺ concentration can be measured, using the methods described above. Furthermore, force generation by hearts or heart tissue can be measured using methods described in Strauss et al., Am. J. Physiol., 262:1437-45, 1992, the contents of which are incorporated herein by reference.

What is claimed is:

1. A method for augmenting SERCA2 mediated calcium ion transport into the sarcoplasmic reticulum of cardiac myocytes of a host comprising: administering to host cardiac myocytes a zinc finger protein that induces expression of SERCA2a, wherein expression of SERCA2a produces an augmentation in SERCA2 mediated calcium ion transport in treated myocytes, as compared to untreated myocytes.

2. The method of claim 1, wherein SERCA2a is expressed from an endogenous SERCA2a gene.

3. The method of claim 1, wherein the zinc finger protein binds to a target nucleotide sequence of a nucleic acid that modulates expression of a SERCA2a gene.

4. The method of claim 1, wherein the zinc finger protein binds to a target nucleotide sequence within the promoter region of a SERCA2a gene.

5. The method of claim 1, wherein the zinc finger protein comprises at least three zinc fingers.

6. The method of claim 1, wherein the zinc finger protein is fused to an effector domain.

7. The method of claim 6, wherein the effector domain is a transcription factor or transcription activation domain thereof.

8. The method of claim 7, wherein the transcription factor or transcription activation domain thereof is selected from the group consisting of p53, NFAT, NF-κB and VP16.

9. The method of claim 1, wherein the zinc finger protein is administered as a nucleic acid encoding the zinc finger protein.

10. The method of claim 9, wherein the nucleic acid is contained within an expression vector.

11. The method according to claim 10, wherein the expression vector is a viral expression vector.

12. The method according to claim 11, wherein the viral expression vector is an adenoviral expression vector or an AAV expression vector.

13. The method according to claim 12, wherein the AAV expression vector is a double-stranded AAV expression vector.

14. The method 9, wherein said nucleic acid is administered in the form of a plasmid.

15. The method of claim 1, further comprising administering an expression vector comprising a polynucleotide encoding SERCA2a.

16. A method for treating or preventing a cardiovascular disorder in a patient comprising: administering to the cardiac muscle of the patient a zinc finger protein that induces expression of SERCA2a, wherein expression of SERCA2a produces an augmentation in SERCA2 mediated calcium ion transport in treated cardiac muscle, thereby increasing cardiac muscle contractility and treating or preventing the cardiovascular disorder.

17. The method of claim 16, wherein SERCA2a is expressed from an endogenous SERCA2a gene.

18. The method of claim 16, wherein the zinc finger protein binds to a target nucleotide sequence of a nucleic acid that modulates expression of a SERCA2a gene.

19. The method of claim 16, wherein the zinc finger protein binds to a target nucleotide sequence within the promoter region of a SERCA2a gene.

20. The method of claim 16, wherein the zinc finger protein comprises at least three zinc fingers.

21. The method of claim 16, wherein the zinc finger protein is fused to an effector domain.

22. The method of claim 21, wherein the effector domain is a transcription factor or transcription activation domain thereof.

23. The method of claim 22, wherein the transcription factor or transcription activation domain thereof is selected from the group consisting of p53, NFAT, NF-κB and VP16.

24. The method of claim 16, wherein the zinc finger protein is administered as a nucleic acid encoding the zinc finger protein.

25. The method of claim 24, wherein the nucleic acid is contained within an expression vector.

26. The method according to claim 24, wherein the expression vector is a viral expression vector.

27. The method according to claim 25, wherein the viral expression vector is an adenoviral expression vector or an AAV expression vector.

28. The method according to claim 16, wherein the AAV expression vector is a double-stranded AAV expression vector.

29. The method 24, wherein the nucleic acid is administered in the form of a plasmid.

30. The method of claim 16, further comprising administering an expression vector comprising a polynucleotide encoding SERCA2a.

31. The method of claim 16, wherein the cardiovascular disorder is selected from the group consisting of restenosis, pulmonary hypertension, heart failure, ischemia, myocardial infarction, congestive heart failure, arrhythmia, and transplant rejection.

32. The method of claim 16, wherein the cardiovascular disorder is a cardiac disorder.

33. The method of claim 31, wherein the cardiac disorder is heart failure.

34. An isolated zinc finger protein comprising: at least three zinc fingers that bind to a region of a SERCA2a gene promoter and induce transcription of the SERCA2a gene.

35. The isolated zinc finger protein of claim 28, further comprising a transcription factor or transcription activation domain thereof.

36. The isolated zinc finger protein of claim 30, wherein the transcription factor or transcription activation domain thereof is selected from the group consisting of p53, NFAT, NF-κB and VP16.

37. An isolated nucleic acid molecule encoding the zinc finger protein of any of claims 34-36.

38. An isolated expression vector comprising the nucleic acid molecule of claim 37.

39. The expression vector of claim 38, wherein the expression vector is a viral expression vector.

40. The expression vector of claim 34, wherein the viral expression vector is an adenoviral expression vector or an AAV expression vector.

41. The expression vector of claim 40, wherein the AAV expression vector is a double-stranded AAV expression vector.


SEQUENCE LISTING (3 sequences)

SEQ ID NO: 1 - protein, 4 aa, Artificial Sequence
  Synthetic construct
CCHH

SEQ ID NO: 2 - protein, 25 aa, Artificial Sequence
  Synthetic construct
CXXXXCXXXXXXXXXXXXHXXXXXH

SEQ ID NO: 3 - protein, 5 aa, Artificial Sequence
  Synthetic construct
TGEKP